(19) United States
(12) Patent Application Publication
(10) Pub. No.: US 2021/0260335 A1
(43) Pub. Date: Aug. 26, 2021

(54) MEDICAL THERAPY SYSTEMS AND METHODS OF USING THE SAME

(71) Applicant: Boston Scientific Scimed, Inc., Maple Grove, MN (US)

(72) Inventors: Martin BURKE, Galway (IE); Daniel TUCK, Galway (IE)

(73) Assignee: Boston Scientific Scimed, Inc., Maple Grove, MN (US)

(21) Appl. No.: 17/185,685

(22) Filed: Feb. 25, 2021

Related U.S. Application Data
(60) Provisional application No. 62/981,649, filed on Feb. 26, 2020.

(51) Int. Cl. A61M 25/00; A61M 1/00

(52) U.S. Cl. CPC A61M 25/0074; A61M 1/0074; A61M 2025/0073; A61M 25/0023; A61M 2025/0024; A61M 1/0023

(57) ABSTRACT

A medical device that includes a shaft defining a lumen and a distal portion, the distal portion defining a side opening in fluid communication with the lumen. The medical device includes a device coupled to the distal portion of the shaft and overlying the side opening. The device includes an expandable member configured to expand laterally outward from a collapsed state to an expanded state, and configured to permit fluid to flow through the expandable member and into the side opening. The device includes a constricting device positioned in contact with the expandable member and configured to move relative to the expandable member from a first position to a second position. The expandable member is in the collapsed state when the constricting device is in the first position and the expandable member is in the expanded state when the constricting device is in the second position.

### CROSS-REFERENCE TO RELATED APPLICATIONS

This application claims the benefit of priority from U.S. Provisional Application No. 62/981,649, filed on Feb. 26, 2020, which is incorporated by reference herein in its entirety.

### TECHNICAL FIELD

Various aspects of the disclosure relate generally to medical vacuum therapy systems, devices, and related methods. Examples of the disclosure relate to systems, devices, and related methods for removing materials from a target site within a patient by generating negative pressure therein, among other aspects.

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical leaks, or other wounds of the tract. Limited treatment options exist for managing such wounds, which have significant morbidity and mortality rates. Options include surgical re-operation and endoscopic placement of a stent or clips. Surgery is relatively invasive and also has high morbidity and mortality rates. Endoscopic stent placement is a less invasive option. The placed stent, however, can migrate from the intended location and/or wall off infection at the treatment site, inhibiting drainage.

### SUMMARY

Aspects of the disclosure relate to, among other things, systems, devices, and methods for treating a target treatment site using negative pressure with an expandable member, among other aspects. Each of the aspects disclosed herein may include one or more of the features described in connection with any of the other disclosed aspects.

According to an example, a medical device includes a shaft defining a lumen and a distal portion, the distal portion defining a side opening in fluid communication with the lumen. The medical device includes a device coupled to the distal portion of the shaft and overlying the side opening. The device includes an expandable member configured to expand laterally outward from a collapsed state to an expanded state, and configured to permit fluid to flow through the expandable member and into the side opening. The device includes a constricting device positioned in contact with the expandable member and configured to move relative to the expandable member from a first position to a second position. The expandable member is in the collapsed state when the constricting device is in the first position and the expandable member is in the expanded state when the constricting device is in the second position.

Any of the medical devices described herein may include one or more of the following features. The constricting device includes an outer tube disposed over the shaft, and the expandable member is enclosed between the shaft and the outer tube when the outer tube is in the first position. The constricting device applies a constricting force against the expandable member when the constricting device is in the first position, and wherein the constricting force is removed when the constricting device is in the second position. The constricting device includes an inner tube disposed within the lumen of the shaft, and includes a distal tip that extends distally of a distal end of the shaft. A proximal portion of the distal tip is coupled to a distal portion of the expandable member. Application of a proximal force on the inner tube causes the expandable member to shorten longitudinally and expand laterally outward. The shaft includes a slot proximate to the distal portion of the shaft and extending into the lumen of the shaft. The constricting device includes a thread extending outwardly from the lumen of the shaft via the slot, and the thread is attached to the distal portion. A proximal portion of the thread extends through the lumen and a distal portion of the thread is disposed over an outer surface of the distal portion of the shaft and extends about the expandable member. The distal portion of the thread includes one or more knots linking the thread to the expandable member. The one or more knots are configured to be broken or untangled in response to a proximal force applied to the thread. Further including a locking mechanism configured to fix the constricting device relative to the expandable member to thereby maintain the constricting device in at least one of the first position or the second position and the expandable member in at least one of the expanded state or the collapsed state. Further including a pressure source in fluid communication with the lumen. The pressure source is configured to generate suction at the distal portion of the shaft via the side opening such that fluid positioned adjacent to the distal portion are drawn into the lumen through the expandable member. The expandable member includes a porous body including a plurality of fibers. The expandable member has a greater diameter in the expanded state than the collapsed state. The expandable member is fixed to the shaft along an entire length of the expandable member.

According to another example, a medical device includes a shaft defining a lumen along a longitudinal axis, wherein the shaft includes at least one opening through an outer surface of the shaft. The at least one opening in fluid communication with the lumen. The medical device includes a porous body disposed over the at least one opening of the shaft and configured to expand laterally outward relative to the longitudinal axis. The medical device includes a constricting member engaged against the porous body and movable relative to the porous body, wherein the constricting member is configured to apply a constraining force against the porous body. The shaft is configured to receive fluid into the lumen via the opening and through the porous body after the constricting member moves the porous body from a collapsed state to an expanded state.

Any of the medical devices described herein may include one or more of the following features. The porous body has a smaller cross-sectional diameter about the longitudinal axis when in the collapsed state than when in the expanded state. The porous body is a sponge having a porosity that allows passage of fluid through the porous body.

According to another example, a method of withdrawing fluids with a medical device, with the medical device including a shaft, an expandable porous body disposed along the shaft, and a constricting device in contact with the expandable porous body, includes moving the constricting device relative to the expandable porous body to allow expansion of the expandable porous body about the shaft. The method includes applying suction through the shaft and withdrawing fluid through the expandable porous body and into the shaft via one or more openings disposed between the expandable porous body and the shaft.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a perspective view of an exemplary medical system including an outer tube and an inner tube, according to aspects of this disclosure;

FIG. 2A is a cross-sectional side view of the inner tube disposed within the outer tube of FIG. 1, with the inner tube including an absorbent device, according to aspects of this disclosure;

FIG. 2B is a cross-sectional side view of the inner tube extending out of the outer tube of FIG. 1 with the absorbent device in an expanded state, according to aspects of this disclosure;

FIG. 3 is a schematic view of the outer tube of FIG. 1 positioned at a target site of a subject, according to aspects of this disclosure;

FIG. 4A is a cross-sectional side view of the inner tube of FIG. 1 disposed within another exemplary outer tube including an absorbent device, according to aspects of this disclosure;

FIG. 4B is a cross-sectional side view of the outer tube of FIG. 4A moving relative to the inner tube with the absorbent device in an expanded state, according to aspects of this disclosure;

FIG. 5 is a partial perspective view of another exemplary inner tube including a constricting device and an absorbent member in a collapsed state, according to aspects of this disclosure;

FIG. 6 is a partial perspective view of the inner tube of FIG. 5 with the constricting device and the absorbent member in an expanded state, according to aspects of this disclosure;

FIG. 7A is a cross-sectional side view of the inner tube of FIG. 5 with the constricting device and the absorbent member in a collapsed state, according to aspects of this disclosure; and

FIG. 7B is a cross-sectional side view of the inner tube of FIG. 5 with the constricting device and the absorbent member in an expanded state, according to aspects of this disclosure.

### DETAILED DESCRIPTION

Endoluminal vacuum therapy (EVAC) has been proposed. In EVAC, negative pressure is delivered to the wound site in the GI tract, for example through a nasogastric tube having a sponge at its terminal end. The sponge is placed endoscopically into the perforation, leak, or other wound. Negative pressure then is applied. Devices and systems suited for EVAC are limited, however.

Examples of the disclosure include systems, devices, and methods for removing materials from a target site within a subject (e.g., patient) by generating a negative pressure therein. Embodiments of this disclosure include devices, systems, and methods for endoluminal vacuum therapy (EVAC). In examples, EVAC includes endoluminal placement of a porous body, e.g., a sponge or other like material into the wound site, including a perforation, a cyst, a leak, an anastomosis, etc. Placement of the material may be via a catheter, scope (endoscope, bronchoscope, colonoscope, etc.), tube, or sheath, inserted into the GI tract via a natural orifice. The orifice can be, for example, the nose, mouth, or anus, and the placement can be in any portion of the GI tract, including the esophagus, stomach, duodenum, large intestine, or small intestine. Placement also can be in other organs reachable via the GI tract.

Reference will now be made in detail to aspects of the disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the subject. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may be used to treat a target site experiencing a leak by negative pressure wound therapy. For example, some embodiments may combine a delivery tube with a porous body and/or an absorbent device to remove any materials (e.g., fluids, masses, etc.) from the target site and dry the surrounding tissue of any leaks. A porous body and/or absorbent device may be positioned along a body of the delivery tube and, in some examples, in fluid communication with a lumen of the delivery tube. A porous body and/or absorbent device may be selectively expandable between a compressed, default state to an expanded, actuated state. Further, a porous body and/or absorbent device may be constricted to the compressed, default state by one or more other components, including, for example, an outer sheath disposed over the absorbent device, a length of the delivery tube, and/or a constricting device coupled to a porous body and/or absorbent device. A porous device and/or absorbent device may be configured to extract matter from within the target site when in the expanded, actuated state.

Examples of the disclosure may relate to devices and methods for performing various medical procedures and/or treating portions of the large intestine (colon), small intestine, cecum, esophagus, any other portion of the gastrointestinal tract, and/or any other suitable patient anatomy (collectively referred to herein as a “target treatment site”). Various examples described herein include single-use or disposable medical devices. Reference will now be made in detail to examples of the disclosure described above and illustrated in the accompanying drawings. Wherever possible, the same reference numbers will be used throughout the drawings to refer to the same or like parts.

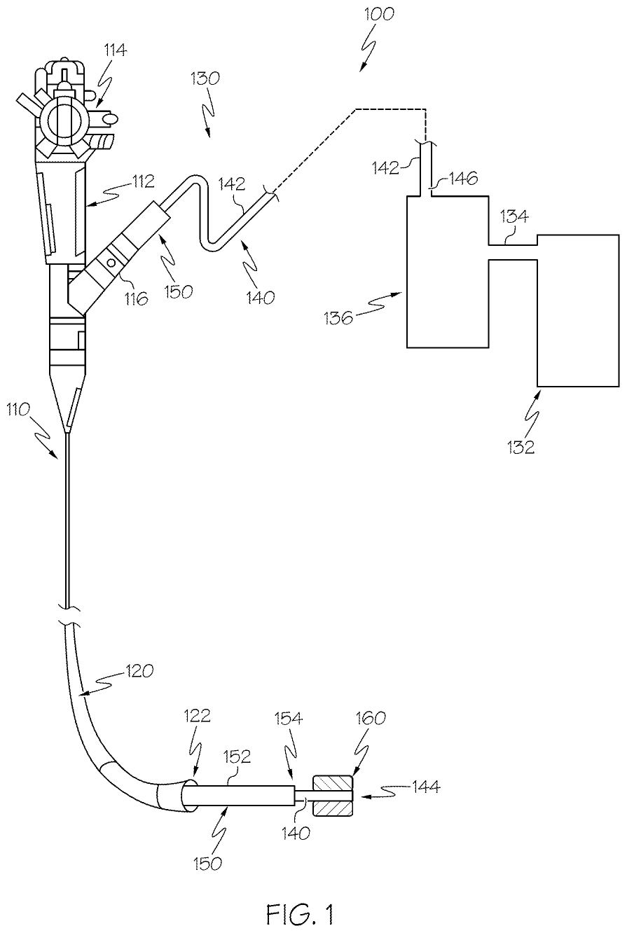

FIG. 1 shows a schematic depiction of an exemplary medical system 100 in accordance with an example of this disclosure. The medical system 100 may include a medical instrument 110, a medical device 130, a pressure regulator 132, and a collection vessel 136. The medical instrument 110 may be configured to facilitate positioning one or more components of the medical system 100 relative to a subject (e.g., a patient), such as, for example, the medical device 130. In embodiments, the medical instrument 110 may be any type of endoscope, duodenoscope, gastroscope, colonoscope, ureteroscope, bronchoscope, catheter, or other delivery system, and may include a handle 112, an actuation mechanism 114, at least one port 116, and a shaft 120. The handle 112 of the medical instrument 110 may have one or more lumens (not shown) that communicate with a lumen(s) of one or more other components of the medical system 100. The handle 112 further includes the at least one port 116 that opens into the one or more lumens of the handle 112. As described in further detail herein, the at least one port 116 is sized and shaped to receive one or more instruments therethrough, such as, for example, the medical device 130 of the medical system 100.

The shaft 120 of the medical instrument 110 may include a tube that is sufficiently flexible such that the shaft 120 is configured to selectively bend, rotate, and/or twist when being inserted into and/or through a subject's tortuous anatomy to a target treatment site. The shaft 120 may have one or more lumens (not shown) extending therethrough that include, for example, a working lumen for receiving instruments (e.g., the medical device 130). In other examples, the shaft 120 may include additional lumens such as a control wire lumen for receiving one or more control wires for actuating one or more distal parts/tools (e.g., an articulation joint, an elevator, etc.), a fluid lumen for delivering a fluid, an illumination lumen for receiving at least a portion of an illumination assembly (not shown), and/or an imaging lumen for receiving at least a portion of an imaging assembly (not shown).

Still referring to FIG. 1, the medical instrument 110 may further include a tip 122 at a distal end of the shaft 120. In some embodiments, the tip 122 may be attached to the distal end of the shaft 120, while in other embodiments the tip 122 may be integral with the shaft 120. For example, the tip 122 may include a cap configured to receive the distal end of the shaft 120 therein. The tip 122 may define one or more openings that are in communication with the one or more lumens of the shaft 120. For example, the tip 122 may include a working opening through which the medical device 130 may exit from a working lumen of the shaft 120.

In other examples, the tip 122 of the shaft 120 may include additional and/or fewer openings thereon, such as, for example, a fluid opening or nozzle through which fluid may be discharged from a fluid lumen of the shaft 120, an illumination opening/window through which light may be emitted, and/or an imaging opening/window for receiving light used by an imaging device to generate an image. The actuation mechanism 114 of the medical instrument 110 is positioned on the handle 112 and may include one or more knobs, buttons, levers, switches, and/or other suitable actuators. The actuation mechanism 114 is configured to control at least one of deflection of the shaft 120 (e.g., through actuation of a control wire), delivery of a fluid, emission of illumination, and/or various imaging functions.

Still referring to FIG. 1, the medical device 130 of the medical system 100 may include a catheter (e.g., multi-lumen catheter) having a longitudinal body extending between a distal end and a proximal end. In the example, the medical device 130 includes an inner tube 140 and an outer tube 150, with the inner tube 140 disposed within and coaxially aligned with the outer tube 150. In the example, a longitudinal body 142 of the inner tube 140 may be longer than a longitudinal body 152 of the outer tube 150. For example, in some embodiments, the longitudinal body 142 of the inner tube 140 may be twice the length of the longitudinal body 152 of the outer tube 150, although other relative lengths are contemplated (e.g., 4x, 5x, or more).

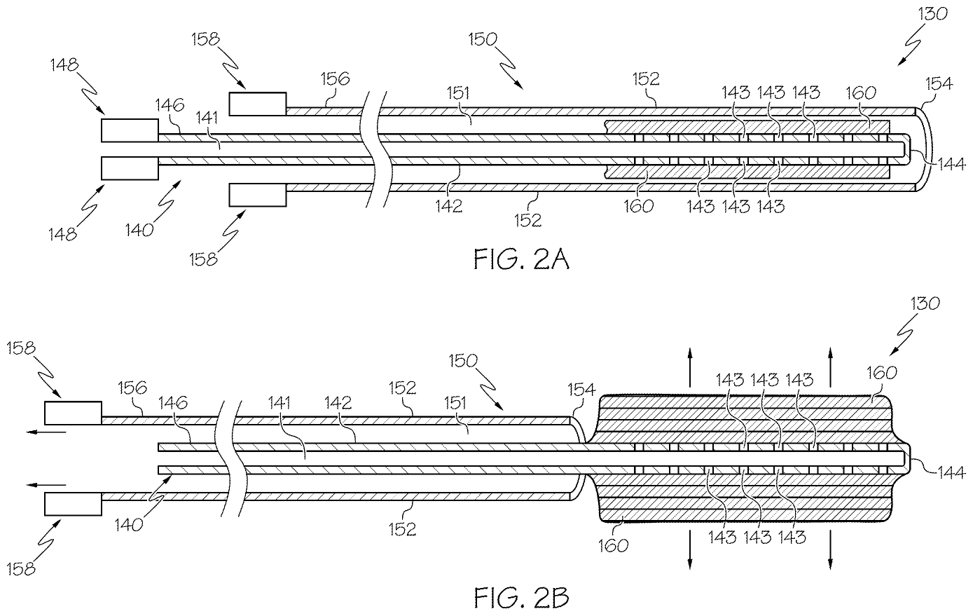

The longitudinal bodies 142, 152 are flexible such that the medical device 130 is configured to bend, rotate, and/or twist when being inserted into a working lumen of the medical instrument 110. As described in detail herein, the medical device 130 may include one or more hubs 148, 158 (FIGS. 2A-2B) attached and adjacent to the proximal end 146, 156, respectively, for actuating the inner tube 140 and the outer tube 150 relative to one another. For example, a hub 148, 158 of the medical device 130 may be configured to move, rotate, and bend the longitudinal bodies 142, 152 of the tubes 140, 150 and/or to move the tubes 140, 150 relative to one another. The hub 148 may be configured and operable to control the longitudinal body 142 of the inner tube 140 and the hub 158 may be configured and operable to control the longitudinal body 152 of the outer tube 150.

Still referring to FIG. 1, the proximal end 146 may define one or more ports (not shown) sized to receive one or more tools through the longitudinal body 142. The proximal end 146 of the inner tube 140 may be fluidly coupled to the collection vessel 136 of the medical system 100 such that the inner tube 140 and/or the outer tube 150 may be in fluid communication with the collection vessel 136. In this instance, a lumen of the inner tube 140 may be fluidly coupled to the collection vessel 136 at the proximal end 146. The collection vessel 136 of the medical system 100 may define a cavity that is sized, shaped, and configured to receive one or more materials from the medical device 130. As described in greater detail herein, the collection vessel 136 may be configured to store materials extracted by the medical device 130 at the distal end 144.

The collection vessel 136 of the medical system 100 may be fluidly coupled to one or more other components of the medical system 100, such as, for example, the pressure regulator 132. In the example, the pressure regulator 132 may be in fluid communication with the collection vessel 136 via a connection port 134 disposed therebetween. The pressure regulator 132 may be configured and operable to generate a negative pressure through the medical system 100. In this instance, the pressure regulator 132 (e.g., a pressurized cylinder) may form a vacuum through the medical system 100 via the collection vessel 136. A lumen of the inner tube 140 and the distal end 144 may become pressurized via the proximal end 146 of the inner tube 140. As described in greater detail herein, the pressure regulator 132 may generate a pressure change through the medical device 130 to extract one or more materials (e.g., fluid) adjacent and/or exterior to the distal end 144, through the inner tube 140, and into the collection vessel 136.

The medical instrument 110 is configured to receive the medical device 130 via the at least one port 116, through the shaft 120 via a working lumen, and to a working opening at the tip 122. In this instance, the medical device 130 may extend distally out of the working opening at the tip 122 and into an exterior environment surrounding the tip 122, such as, for example, at a target treatment site of a subject as described in further detail below. The outer tube 150 may extend distally from the working opening of the tip 122 in response to a translation of the longitudinal body of the medical device 130 through a working lumen of the shaft 120.

Additionally, the inner tube 140 of the medical device 130 may be disposed within a lumen of the outer tube 150 and thereby extend distally from the working opening of the tip 122 with the outer tube 150 in response to translation of the medical device 130 through a working lumen of the shaft 120 (i.e., the tubes 140, 150 may move simultaneously). In this instance, it should be appreciated that the inner tube 140 may be disposed within a lumen of the outer tube 150 such that the distal end 144 of the inner tube 140 is positioned proximally relative to a distal end 154 of the outer tube 150.

Still referring to FIG. 1, the medical device 130 may further include a device at the distal end 144 of the inner tube 140. In the example, the device of the medical device 130 may include an expandable member 160. The expandable member 160 may be positioned at and/or extend distally relative to the distal end 144 of the inner tube 140. In some embodiments, the expandable member 160 may include an absorbent device coupled to the distal end 144 of the inner tube 140. In the example, the expandable member 160 may be positioned along a sidewall and/or exterior surface of the longitudinal body 142 of the inner tube 140.

It should be appreciated that, in other embodiments, the expandable member 160 may be positioned along various other surfaces of the inner tube 140 and/or portions of the medical device 130 without departing from a scope of this disclosure. A majority or entirety of the expandable member 160 may be positioned proximally to a distalmost portion of the distal end 144 of the inner tube 140. In other embodiments, at least a portion and/or an entirety of the expandable member 160 may extend distally relative to the distal end 144 of the inner tube 140.

Referring now to FIG. 2A, the expandable member 160 may include a porous body including a plurality of flexible and/or compactible fibers. In embodiments of this disclosure, the porous body may include a sponge and/or foam that may be any suitable biocompatible material capable of absorbing liquids and/or permitting liquid to pass therethrough via negative pressure, such as, for example, from the pressure regulator 132 of the medical system 100. The material of the porous body (e.g., the plurality of fibers) of the expandable member 160 may be flexible, compressible, porous, hydrophilic, sterile, and/or disposable. Further, the sponge material of the porous body may be an open-cell foam. Suitable materials may include, for example, polyurethanes, esters, ethers, composite materials, absorbing polymer powder, micro/nano-porous materials, and any medical-grade material.

In the example, the porous body of the expandable member 160 may define a mesh formed by the plurality of fibers. In this instance, the expandable member 160 may be configured and operable to selectively expand. In other words, a volume capacity of the expandable member 160 may be selectively adjusted. It should be appreciated that, with the expandable member 160 forming a sponge and/or foam, the expandable member 160 may be configured to permit passage and/or delivery of one or more materials (e.g. fluid) through the porous body of the expandable member 160.

Still referring to FIG. 2A, and as described above, the medical device 130 includes one or more hubs 148, 158 (e.g., handles) for manually grasping and actuating the tubes 140, 150. In the example, the inner tube 140 may include the proximal hub 148 along the longitudinal body 142 at and/or proximate to the proximal end 146 of the inner tube 140; and the outer tube 150 may include the proximal hub 158 along the longitudinal body 152 at and/or proximate to a proximal end 156 of the outer tube 150. In the example, the proximal hubs 148, 158 of the tubes 140, 150 may be coupled to the longitudinal bodies 142, 152, respectively. As described further herein, one or more of the proximal hubs 148, 158 may include a luer capable of coupling the tubes 140, 150 to the collection vessel 136 and/or the pressure regulator 132. In some embodiments, the proximal hub 148, 158 may be selectively removable from the longitudinal bodies 142, 152 of the tubes 140, 150, respectively. For example, the proximal hub 148 of the inner tube 140 may be releasably coupled to the proximal end 146 of the longitudinal body 142 such that the proximal hub 148 may be configured to decouple from the inner tube 140.

In the example, the longitudinal body 142 of the inner tube 140 may define a lumen 141 extending between the distal end 144 and the proximal end 146. As described in detail above, the proximal end 146 of the inner tube 140 may be fluidly coupled to the collection vessel 136 and the pressure regulator 132 via the proximal hub 148, such that the lumen 141 of the inner tube 140 is in fluid communication thereto. The distal end 144 of the inner tube 140 may be closed thereby terminating the lumen 141 of the longitudinal body 142 therein. In other words, in some embodiments, fluid cannot be conveyed through the distal end 144. However, in other embodiments, it is contemplated that the distal end 144 of the inner tube 140 may be open thereby permitting conveyance of fluid through the distal end 144.

Further, the longitudinal body 152 of the outer tube 150 may define a lumen 151 extending between the distal end 154 and a proximal end 156 of the outer tube 150. The distal end 154 and the proximal end 156 of the outer tube 150 may each form an opening that is sized, shaped, and configured to slidably receive the longitudinal body 142 of the inner tube 140 therethrough. In this instance, the inner tube 140 may be received within the lumen 151 of the outer tube 150 via the opening at the proximal end 156; and may extend distally and/or outward from the lumen 151 via the opening at the distal end 154 of the outer tube 150.

Still referring to FIG. 2A, the inner tube 140 of the medical device 130 may further include one or more openings 143 disposed along the outer walls of the longitudinal body 142 and adjacent to the distal end 144. In the example, the inner tube 140 may include a plurality of openings 143 extending along a distal portion of the longitudinal body 142 that is longitudinally coincident with a position of the expandable member 160 on the longitudinal body 142. The plurality of openings 143 may extend through the longitudinal body 142 and into the lumen 141 of the inner tube 140. With the expandable member 160 disposed along an exterior of the longitudinal body 142 and positioned over the plurality of openings 143, it should be appreciated that a porous body of the expandable member 160 may be in fluid communication with the lumen 141 of the inner tube 140 via the plurality of openings 143. The openings 143 may include various suitable sizes, shapes, and/or configurations. In some embodiments, the openings 143 of the inner tube 140 may be micron-sized (e.g., micrometer), although other suitable dimensions are also contemplated (e.g., millimeter, centimeter, etc.).

With the inner tube 140 disposed within the lumen 151 of the outer tube 150, it should be appreciated that an interior surface of the longitudinal body 152 may be operable to constrict a size, shape, profile, and/or configuration of the expandable member 160. In other words, the outer tube 150 may be a constricting device configured to contact and/or engage the expandable member 160 to thereby collapse a porous body of the expandable member 160 to a compressed state. As shown and described in further detail herein, the medical device 130 may be configured such that removal of the outer tube 150 over the expandable member 160 may cease application of a constricting/constraining force against the expandable member 160 by the longitudinal body 152 and allow a porous body of the expandable member 160 to expand outwardly (FIG. 2B) to its resting/biased state.

Referring now to FIG. 2B, the inner tube 140 is schematically depicted with the proximal hub 148 omitted from the proximal end 146. As briefly described above, the proximal hub 148 may be a removable luer and/or hub such that the proximal hub 148 may be configured to decouple from the longitudinal body 142. In this instance, removal of the proximal hub 148 from the longitudinal body 142 may permit a proximal translation of the outer tube 150 relative to the inner tube 140. It should be appreciated that inclusion of the proximal hub 148 along the proximal end 146 of the longitudinal body 142 may inhibit a proximal translation of the outer tube 150 beyond the proximal end 146 of the inner tube 140 due to a presence of the proximal hub 148 thereon. In other words, it should be understood that the proximal hub 148 of the inner tube 140 may engage and/or abut against the proximal hub 158 of the outer tube 150 when translating the outer tube 150 proximally, thereby securing the outer tube 150 to the inner tube 140 (and effectively acting as a stop).

With the proximal hub 148 of the inner tube 140 decoupled from the proximal end 146 of the longitudinal body 142, the outer tube 150 may translate proximally to an extent such that the expandable member 160 may extend outwardly from the lumen 151 of the longitudinally body 152. In this instance, a constricting force applied to the expandable body 160 by the outer tube 150 may be removed, thereby allowing the expandable body 160 to expand radially, proximally, and/or distally outwardly relative to the inner tube 140. The expandable body 160 may extend laterally outward relative to, and about, the longitudinal body 142 such that the expandable body 160 has a larger diameter when in the expanded state than in the collapsed state (FIG. 2A).

Still referring to FIG. 2B, it should be understood that the expandable body 160 may remain secured to the longitudinal body 142 of the inner tube 140 when the expandable body 160 is in the expanded state. In the example, an entire length of the porous body of the expandable member 160 is fixed to the longitudinal body 142 and maintained over the plurality of openings 143 when transitioning from the collapsed state (FIG. 2A) to the expanded state. Accordingly, the porous body of the expandable member 160 may remain in fluid communication with the lumen 141 of the inner tube 140 via the plurality of openings 143 when extended to the expanded state. As described in greater detail herein, the plurality of openings 143 may be configured and operable to extract or allow one or more materials (e.g., solid matter, fluid, etc.) within or exterior of the porous body to flow through the porous body of the expandable member 160 and into the lumen 141 of the inner tube 140 (via the plurality of openings 143) when the expandable member 160 is in an expanded state.

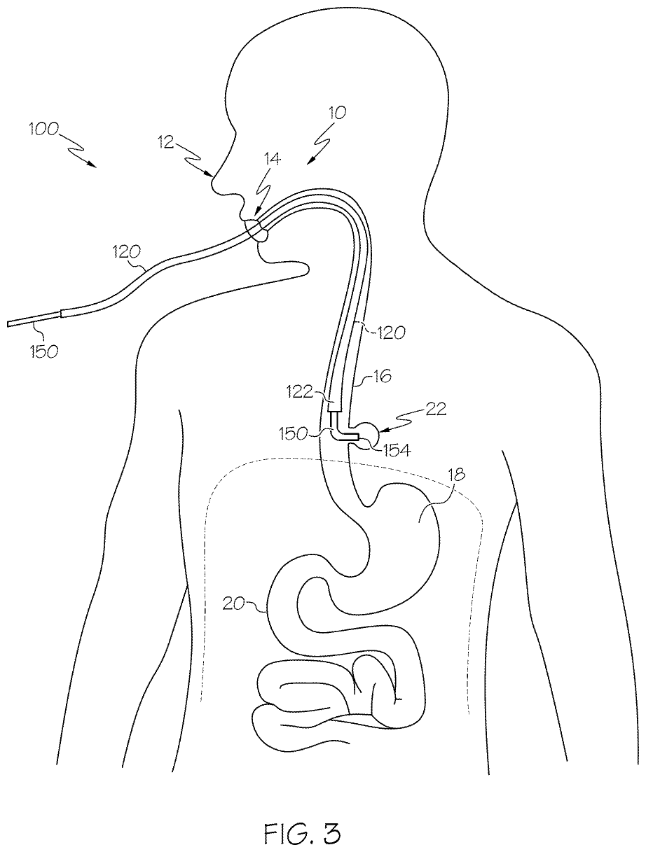

Referring now to FIG. 3, an exemplary method of using the medical system 100 to treat a target site within a subject 10 (e.g., patient) is schematically depicted. For example, the shaft 120 of the medical instrument 100 may be guided through a digestive tract of the subject 10 by inserting the tip 122 into a nose 12 or mouth 14 (or other suitable natural body orifice) of the subject's body 10. In embodiments, the medical instrument 110 may be inserted through a gastrointestinal tract of the subject's body 10, including an esophagus 16, a stomach 18, and/or into an intestinal tract 20 (e.g., small intestine, large intestine) until reaching a target treatment site. It should be appreciated that a length of the shaft 120 may be sufficient so that a proximal end of the medical instrument 110 (including the handle 112) is external of the subject's body 10 while the tip 122 of the medical instrument 110 is internal to the subject's body 10.

In the example, a target treatment site 22 within the subject's body 10 may be located along the gastrointestinal tract within the esophagus 16. The target treatment site 22 may include an anastomotic leak or perforation in the esophagus 16 or other portions of the GI tract, such as, for example, a wound resulting from a surgical procedure performed therein (e.g., colonic resection, bariatric surgery, esophagectomy, etc.). While this disclosure relates to the use of the medical system 100 in a digestive tract of the subject's body 10, it should be understood that the features of this disclosure could be used in various other locations (e.g., other organs, tissue, etc.) within the subject's body 10. In some examples, a shape and/or size of a porous body of the expandable member 160 may be adjusted (e.g., manually trimmed) by a user prior to deployment of the medical device 130 into the subject 10.

Still referring to FIG. 3, the shaft 120 of the medical instrument 110 may extend into the subject's body 10 until it reaches a position in which the tip 122 of the shaft 120 is located proximate to the target treatment site 22 (e.g., anastomotic leak or perforation). It should be understood that the medical device 130 (e.g., the tubes 140, 150) may be received within the medical instrument 100, such as, for example, within the shaft 120 through the port 116, prior to and/or after insertion of the medical instrument 110 into the subject's body 10. With the tip 122 of the shaft 120 positioned adjacent to the target treatment site 22 and the medical device 130 disposed therein, the medical device 140 may be actuated (e.g., at the proximal hubs 148, 158) to extend the outer tube 150 and/or the inner tube 140 distally from the tip 122 of the shaft 120. In this instance, the distal end 154 of the outer tube 150 extends outwardly from a lumen of the shaft 120.

With the outer tube 150 positioned within the esophagus 16 (or GI tract) and adjacent to the target treatment site 22, a user may actuate one or more components of the medical instrument 110 and/or the medical device 130 to position the distal end 154 of the outer tube 150 within or adjacent to the target treatment site 22 (e.g., anastomotic leak or perforation). For example, the proximal hub 158 (FIGS. 2A-2B) of the medical device 140 may be actuated to articulate (e.g., deflect, bend, pivot, etc.) the distal end 154 of the outer tube 150 relative to the tip 122 of the shaft 120. In this instance, a distal portion of the outer tube 150 (and the inner tube 140 received therein) may be moved into the perforation of the target treatment site 22 with a remainder of the longitudinal body 142, 152 of the tubes 140, 150 positioned external of the target treatment site 22. In this instance, it should be appreciated that the expandable body 160 of the medical device 130 may be positioned within the target treatment site 22 and in the deflated and/or collapsed state (FIG. 2A). Alternatively, the distal end 154 of the outer tube 150 may be positioned adjacent to the target treatment site 22 and withdrawn to expand the expandable member 160. In this instance, the expandable member 160 may be moved into the target treatment site 22.

Still referring to FIG. 3, the shaft 120 of the medical instrument 110 may be withdrawn from the esophagus 16 (GI tract) of the subject's body 10 upon positioning the medical device 130 at the target treatment site 22. With the distal ends 144, 154 of the tubes 140, 150 disposed within the target treatment site 22 (e.g., anastomotic leak or perforation), the proximal ends 146, 156 of the tubes 140, 150 may be positioned external of the subject's body 10. In this instance, the proximal hubs 148, 158 of the tubes 140, 150 may be accessible to a user of the medical system 100. For example, the proximal hub 148 of the inner tube 140 may be removed from the proximal end 146 of the longitudinal body 142 to facilitate a removal of the outer tube 150 over the inner tube 140.

In some embodiments, the inner tube 140 may not be coupled to the pressure regulator 132 during insertion of the inner tube 140 into the esophagus 16 (GI tract). In this instance, after removal of the proximal hub 148, a different hub may be attached to the proximal end 146 to fluidly couple the inner tube 140 to the pressure regulator 132. In other embodiments, the proximal hub 148 may be reattached to the proximal end 146 and be configured to fluidly couple the inner tube 140 to the pressure regulator 132. In some embodiments, removal of the proximal hub 148 from the proximal end 146 of the longitudinal body 142 may decouple a fluid communication between the lumen 141 of the inner tube 140 and the collection vessel 136 and/or the pressure regulator 132. In this instance, proximal retraction of the proximal hub 158 of the outer tube 150 may expose the distal end 144 of the inner tube 140 from the lumen 151, such that the expandable member 160 extends distally from the distal end 154 of the longitudinal body 152.

The porous body of the expandable member 160 may progressively transition from the collapsed state (FIG. 2A) to the expanded state (FIG. 2B) as the outer tube 150 translates proximally from a first position (FIG. 2A) to a second position (FIG. 2B). It should be understood that the inner tube 140 of the medical device 130 may remain at a fixed position relative to the target treatment site 22 as the outer tube 150 is translated. In other embodiments, the inner tube 140 may extend distally relative to the outer tube 150 and/or toward the target treatment site 22.

Referring back to FIG. 2B, with the expandable member 160 in an expanded state and the distal end 144 of the longitudinal body 142 disposed within the target treatment site 22, the porous body of the expandable member 160 may contact the surrounding tissue defining the target treatment site 22 or otherwise be disposed in the target treatment site 22. In this instance, the expandable member 160 may be configured to absorb one or more materials (e.g., fluid, solids, etc.) located in the target treatment site 22. For instance, the porous body of the expandable member 160 may absorb fluids in the target treatment site 22, thereby drying the surrounding tissue defining the target treatment site 22. In some embodiments, the expandable member 160 may be further configured to divert (e.g., push, reposition, move, dislodge, displace, remove, etc.) one or more materials within the target treatment site 22 when extending outward to the expanded state.

Additionally, the expandable member 160 may apply a force and/or generate a pressure within the target treatment site 22, and against any materials disposed therein, in response to an expansion of the porous body of the expandable member 160. In some embodiments, the outer tube 150 may be fully removed from over the inner tube 140 (and from the patient) such that the proximal end 146 of the longitudinal body 142 may be accessible to a user of the medical system 100. In this instance, the proximal hub 148 (and/or another luer/hub connector) may be coupled to the proximal end 146 of the inner tube 140. With the proximal hub 148 coupled to the longitudinal body 142 of the inner tube 140, the collection vessel 136 and/or the pressure regulator 132 may be fluidly coupled to the lumen 141 of the inner tube 140 via the proximal hub 148.

With the lumen 141 of the inner tube 140 fluidly coupled to the pressure regulator 132, the lumen 141 may be configured and operable to convey (e.g., by suction, extraction, pulling, withdrawing, etc.) one or more materials (e.g. fluid, bodily matter, etc.) from the target treatment site 22 to the collection vessel 136 via the expandable body 160 and the plurality of openings 143 in response to actuation of the pressure regulator 132. In the example, the one or more materials disposed in the target treatment site 22 may be moved about and/or through the porous body of the expandable member 160. In other words, a negative pressure is generated within the target treatment site 22 by the pressure regulator 132 via the plurality of openings 143.

The one or more materials disposed within the target treatment site 22 (e.g., leak or perforation) may include solids, liquids, and/or various other matters received therein from the gastrointestinal tract of the subject. It should be understood that a presence of said materials in the leak and/or perforation in the GI tract may inhibit recovery and/or healing of the target treatment site. Accordingly, the one or more materials disposed within a cavity of the target treatment site 22 may be suctioned through a porous body of the expandable member 160 and into the lumen 141 of the inner tube 140.

Referring back to FIG. 1, with the proximal end 146 of the inner tube 140 fluidly coupled to the collection vessel 136, the materials and/or fluid received in the lumen 141 from the target treatment site 22 may be deposited in the collection vessel 136. It should be appreciated that the collection vessel 136 and the pressure regulator 132 are arranged and/or configured such that the pressure regulator 132 may continue to generate a negative pressure in the lumen 141 of the inner tube 140 through the collection vessel 136 as the collection vessel 136 stores one or more materials and/or fluid therein.

For example, the connection port 134 coupling the collection vessel 136 and the pressure regulator 132 may be positioned along a top portion of the collection vessel 136 such that fluid communication therebetween is maintained as a bottom portion of the collection vessel 136 receives the materials and/or fluid therein. In other embodiments, the collection vessel 136 and the pressure regulator 132 may be separated from one another and coupled to the proximal end 146 of the inner tube 140 through various other suitable arrangements than those shown and described herein. Upon completion of a procedure, the pressure regulator 132 may be deactivated to thereby cease a suction within the target treatment site 22 through the inner tube 140.

Still referring to FIG. 1, a withdrawal of the inner tube 140 through the esophagus 16 (GI tract) (e.g., outward from the nose 12 and/or mouth 14) may cause the porous body of the expandable member 160 to adjust (e.g., compress) accordingly, such as, for example, relative to the walls of the esophagus 16 as the inner tube 140 is retracted therethrough. In some embodiments, the outer tube 150 of the medical device 130 may be repositioned over the longitudinal body 142 of the inner tube 140 (e.g. before withdrawal) to thereby enclose the expandable member 160 within the lumen 151 to facilitate a removal of the medical device 130 from the subject 10.

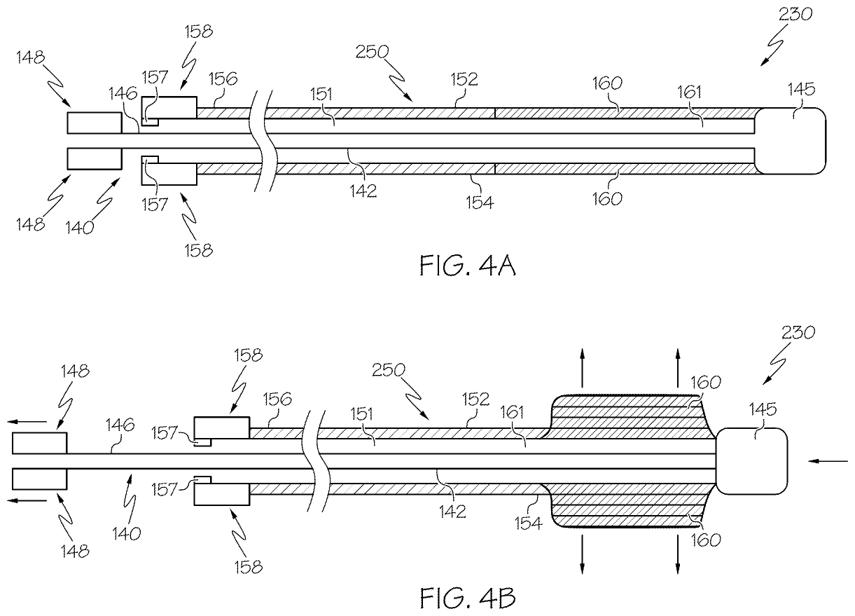

Referring now to FIGS. 4A-4B, another exemplary medical device 230 is depicted in accordance with an example of this disclosure. Except as otherwise described below, the medical device 230 may be substantially similar to the medical device 130 described above such that like reference numerals are used to identify like components. It should be understood that the medical device 230 may be configured and operable like the medical device 130 and that the medical device 230 may be readily incorporated into the medical system 100 described above.

For example, referring initially to FIG. 4A, the medical device 230 may include an outer tube 250 having a longitudinal body 152 defined between a distal end 154 and a proximal end 156. The outer tube 250 may be directly coupled with a proximal end of the expandable member 160, at the distal end 154. In this instance, a proximal end of the expandable member 160 may be secured to the distal end 154 of the outer tube 250 such that the expandable member 160 may be aligned with the longitudinal body 152 of the outer tube 250. In other words, the expandable member 160 may extend distally from the distal end 154 of the outer tube 250 and a longitudinal length of the expandable member 160 is an extension of the longitudinal body 152 of the outer tube 250. In this instance, the expandable member 160 may define a lumen 161 that is aligned with and distal to the lumen 151 of the outer tube 250.

The outer tube 250 may include the proximal hub 158 at the proximal end 156 of the longitudinal body 152. In the example, the proximal hub 158 of the outer tube 250 may include a locking feature 157 disposed along an interior surface of the proximal hub 158 such that the locking feature 157 extends toward and/or at least partially within the lumen 151. As described in further detail herein, the locking feature 157 of the outer tube 250 may be configured and operable to engage the inner tube 140 of the medical device 230 to fix the longitudinal body 152 relative to the longitudinal body 142 of the inner tube 140. In some embodiments, the locking feature 157 may include, for example, a hemostatic valve, a rubber gasket (e.g., an O-ring), and/or various other suitable locking mechanisms.

Still referring to FIG. 4A, the inner tube 140 of the medical device 230 may be disposed within the lumen 151 of the outer tube 250 and the lumen 161 of the expandable member 160. In the example, the inner tube 140 may include a longitudinal body 142 defined between a distal end 145 and a proximal end 146. In the example, the distal end 145 extends distally relative to the distal end 154 of the outer tube 250 and a distal end of the expandable member 160. Further, the distal end 145 of the inner tube 140 may be sized and shaped to have a cross-sectional dimension larger than cross-sectional dimensions of the lumen 151 and the lumen 161. A distal end of the expandable member 160 may be directly coupled and secured to the distal end 145 of the inner tube 140.

In the example shown in FIG. 4A, the distal end 145 of the inner tube 140 may be positioned at a distalmost extent relative to the outer tube 250 (e.g., a first position) such that the expandable member 160 is maintained in a collapsed state. In this instance, a longitudinal body of the expandable member 160 may be stretched between the distal ends 145, 154 of the tubes 140, 250. As described further herein, the expandable member 160 is configured to move relative to the outer tube 250 in response to movement of the inner tube 140 relative to the outer tube 250.

In other words, the distal end 145 of the inner tube 140 may be a constricting device configured to abut against a distal end of the expandable member 160. In this instance, the expandable member 160 may be operable to transition from the collapsed state to the expanded state (FIG. 4B) in response to movement of the inner tube 140 relative to the outer tube 250 (e.g., to a second position). It should be appreciated that a porous body of the expandable member 160 may have a smaller profile, cross-sectional dimension, diameter, etc., and a longer longitudinal length, when in the collapsed state than when in the expanded state.

Referring now to FIG. 4B, with the proximal hub 148 of the inner tube 140 translated proximally relative to the proximal end 156 of the longitudinal body 152, the distal end 145 of the inner tube 140 may translate proximally to an extent (e.g., a second position) such that the expandable member 160 is engaged and longitudinally-collapsed by the distal end 145. In this instance, the expandable member 160 may extend radially outward relative to the longitudinally body 152 of the outer tube 250. In this instance, a constricting force applied to the expandable body 160 by the distal end 145 may be generated, thereby causing the expandable body 160 to expand radially outward. The expandable body 160 may extend laterally/radially outward relative to the distal end 154 of the outer tube 250 such that the expandable body 160 forms a greater profile, cross-sectional dimension, diameter, etc., and a shorter longitudinal length, when in the expanded state than in the collapsed state (FIG. 4A).

It should be understood that the expandable body 160 may remain secured to the distal end 154 of the longitudinal body 152 when in the expanded state. In this instance, proximal translation of the distal end 145 of the inner tube 140 compresses the expandable body 160 between the distal end 154 of the outer tube 250 and the distal end 145 of the inner tube 140. In other words, an entire length of the porous body of the expandable member 160 is sandwiched between the distal ends 145, 154 in response to movement of the inner tube 140 relative to the outer tube 250. In other embodiments, the expandable body 160 may be transitioned to the expanded state in response to a distal translation of the outer tube 250 relative to the inner tube 140.

Still referring to FIG. 4B, with the expandable member 160 in the expanded state the proximal hub 158 of the outer tube 250 may be actuated to lock the outer tube 250 to the inner tube 140 to thereby maintain the expandable member 160 in the expanded state. For example, applying a lateral (e.g., inward) force onto the proximal hub 158 may provide a movement of the proximal hub 158 and/or the distal end 154 of the longitudinal body 152 toward the inner tube 140. In this instance, the locking feature 157 of the proximal hub 158 may contact and engage the longitudinal body 142 of the inner tube 140, thereby fixing a position of the outer tube 250 and the expandable member 160 relative to the inner tube 140. Accordingly, the expandable member 160 may be maintained in the expanded state while the locking feature 157 remains engaged with the inner tube 140.

Although not shown, it should be understood that the inner tube 140 may include the plurality of openings 143 shown and described above (FIGS. 2A-2B) such that the porous body of the expandable member 160 may be in fluid communication with the lumen 141 of the inner tube 140 via the plurality of openings 143. In this instance, the expandable body 160 may be fluidly coupled to the collection vessel 136 and/or the pressure regulator 132 via the lumen 141 of the inner tube 140. In other embodiments, the lumen 151 of the outer tube 250 may be fluidly coupled to the collection vessel 136 and/or the pressure regulator 132. In this instance, the openings 143 of the inner tube 140 may be omitted entirely and the expandable body 160 may be in fluid communication with the collection vessel 136 and/or the pressure regulator 132 via the lumens 151, 161.

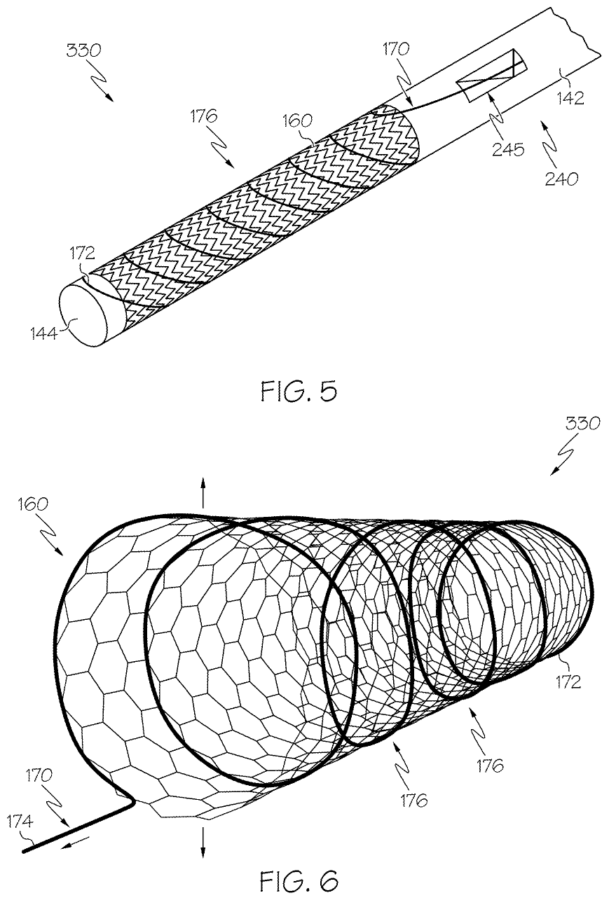

Referring now to FIGS. 5-7B, another exemplary medical device 330 is depicted in accordance with an example of this disclosure. Except as otherwise described below, the medical device 330 may be substantially similar to the medical device 130 described above such that like reference numerals are used to identify like components. It should be understood that the medical device 330 may be configured and operable like the medical device 130 and that the medical device 330 may be readily incorporated into the medical system 100 described above.

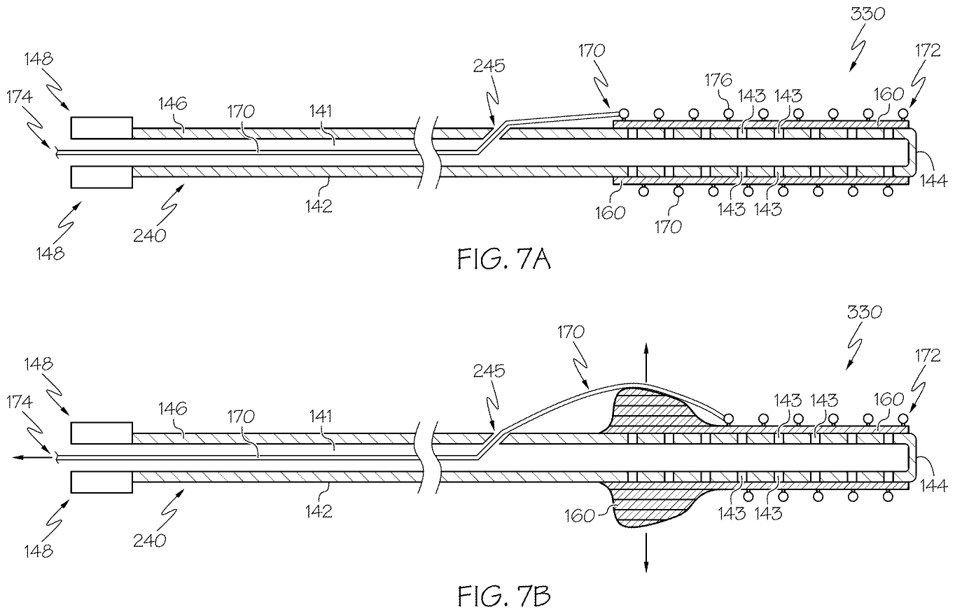

For example, referring initially to FIGS. 5 and 7A, the medical device 330 may include a tube 240 having a longitudinal body 142 defined between a distal end 144 and a proximal end 146. The longitudinal body 142 of the tube 240 may include an opening and/or slot 245 disposed thereon, such as, for example, along an intermediate portion of an outer wall of the longitudinal body 142 and adjacent to the distal end 144. As shown and described in further detail herein, the slot 245 may be sized and shaped to extend into the lumen 141 of the tube 240. In some embodiments, the slot 245 may include a ramped surface for guiding one or more components of the medical device 330 from the lumen 141 of the tube 240 to an exterior surface of the longitudinal body 142 and/or toward the distal end 144.

The tube 240 of the medical device 330 may further include the expandable member 160 disposed along an exterior surface of the longitudinal body 142 and positioned adjacent to the distal end 144. In the example, the expandable member 160 may be disposed about the longitudinal body 142 between the distal end 144 and the slot 245 of the tube 240. The medical device 330 may further include a thread 170 having a longitudinal body defined between a distal end 172 and a proximal end 174. The thread 170 may be at least partially disposed within the lumen 141 of the tube 240 and a distal portion of the thread 170 (e.g., including the distal end 172) may extend outwardly from the lumen 141 via the slot 245. In the example, a distal portion of the thread 170 may extend along an exterior surface of the longitudinal body 142 of the tube 240 from the slot 245 and toward the distal end 144.

Although not shown, it should be appreciated that, in some embodiments, the tube 240 may include multiple channels disposed within the lumen 141 and separated from another. In the embodiment, at least one of the multi-channels in the lumen 141 may be aligned with the slot 245 of the tube 240 and configured to receive the thread 170 therethrough. At least another of the multi-channels in the lumen 141 may be aligned with the plurality of openings 143 for fluidly coupling the pressure regulator 132 and/or the collection vessel 136 thereto. In this instance, the slot 245 (and the thread 170 received therein) may be isolated from the vacuum generated in the tube 240 by the pressure regulator 132.

Still referring to FIGS. 5 and 7A, with the expandable member 160 disposed about a distal portion of the longitudinal body 142 and the thread 170 extending outwardly from the slot 245 and toward the distal end 144, the thread 170 may be configured to extend over a porous body of the expandable member 160. In the example, a longitudinal length of the thread 170 may extend about a circumference of the expandable member 160 thereby forming one or more slip knots 176 (or connecting areas) positioned along a porous body of the expandable member 160. For example, the thread 170 may include a plurality of slip knots 176 encapsulating the expandable member 160 therein when in a first position. A connecting area may be a portion of thread 170 that is releasably or breakably-coupled to the expandable member 160. For example, absent the application of a breaking force on the connecting areas, the connected areas of the thread 170 and the expandable member 160 may remain coupled to one another (e.g., during movement of the expandable member 160).

The plurality of slip knots 176 may be configured to apply a force against the expandable member 160 to constrict movement of the expandable member 160 relative to the tube 240. In other words, the thread 170 may be a constricting device and the plurality of slip knots 176 may be a constricting mechanism securely bound around the expandable member 160 in a constricted configuration such that the expandable member 160 is fixed against the longitudinal body 142. As described in further detail herein, the thread 170 may be configured such that releasing the plurality of slip knots 176 to a loosened configuration may allow the expandable member 160 to transition from the collapsed state to an expanded state (FIGS. 6 and 7B).

Referring to FIG. 6, the medical device 330 is schematically depicted with the longitudinal body 142 of the tube 240 omitted for purposes of clarity. As shown herein, the plurality of slip knots 176 extend about a longitudinal length of the expandable member 160 and terminate at a distalmost slip knot 176 adjacent to the distal end 172 of the thread 170. Further, the proximal end 174 of the thread 170 may include a linear configuration that is manually or mechanically graspable by a user of the medical system 100. Accordingly, actuating the proximal end 174 of the thread 170 (e.g., by applying a proximal, pulling force) may provide a proximal translation of the thread 170 (e.g., from a first position to a second position) and a transition/breaking of one or more of the plurality of slip knots 176 from a constricted configuration (FIG. 5) to a loosened configuration (FIG. 6). In this instance, the expandable member 160 may expand laterally outward to an expanded state.

Referring now to FIG. 7B, in some embodiments, the thread 170 may be configured such that actuation of the proximal end 174 (e.g., to a second position) may provide a removal of one or more of the plurality of slip knots 176. In this instance, a proximalmost slip knot 176 of the thread 170 may be unwound from about a circumference of the expandable member 160, thereby removing a constricting force applied thereto. Upon removal of the constricting force against the expandable member 160 by the thread 170, a porous body of the expandable member 160 may be permitted to expand laterally outward relative to the longitudinal body 142 of the tube 240.

It should be understood that a remaining portion of the expandable member 160 (e.g., a distal length) may remain in a collapsed state despite an actuation of the thread 170 when one or more of the plurality of slip knots 176 are maintained along the porous body of the expandable member 160. In this instance, the expandable member 160 may be at least partially expanded and at least partially collapsed. The expandable member 160 may transition to a fully expanded state in response to removal of all and/or substantially all of the plurality of slip knots 176 of the thread 170 along an exterior of the expandable member 160. The breaking of each individual slip knot 176 may provide tactile feedback to a user of the medical device 330 as to an extent of expansion of the expandable member 160.

Each of the aforementioned systems, devices, assemblies, and methods may be used to provide controlled treatment of a target treatment site via endoluminal vacuum therapy. Any of the medical devices 130, 230, 330, for example, the tubes 140, 150, 240, 250 and the expandable member 160 of the medical devices 130, 230, 330 shown and described above, may be inserted into an endoscope (e.g., medical instrument 110), or like device, with imaging systems, lighting systems, etc., to assist in positioning the medical devices 130, 230, 330. By providing a device that allows a user to treat a difficult-to-reach tissue using an expandable member 160 that applies a negative pressure through a vacuum sealed sponge, a user may reduce overall procedure time, increase efficiency and efficacy of procedures, and avoid unnecessary harm to a subject's body caused by non-healing wounds.

It will be apparent to those skilled in the art that various modifications and variations may be made in the disclosed devices and methods without departing from the scope of the disclosure. It should be appreciated that the disclosed devices may include various suitable computer systems and/or computing units incorporating a plurality of hardware components, such as, for example, a processor and non-transitory computer-readable medium, that allow the devices to perform one or more operations during a procedure in accordance with those described herein. Other aspects of the disclosure will be apparent to those skilled in the art from consideration of the specification and practice of the features disclosed herein. It is intended that the specification and examples be considered as exemplary only.

We claim:

1. A medical device, comprising:
a shaft defining a lumen and a distal portion, the distal portion defining a side opening in fluid communication with the lumen; and
a device coupled to the distal portion of the shaft and overlying the side opening, the device including:
an expandable member configured to expand laterally outward from a collapsed state to an expanded state, and configured to permit fluid to flow through the expandable member and into the side opening; and
a constricting device positioned in contact with the expandable member and configured to move relative to the expandable member from a first position to a second position;
wherein the expandable member is in the collapsed state when the constricting device is in the first position and the expandable member is in the expanded state when the constricting device is in the second position.

2. The medical device of claim 1, wherein the constricting device includes an outer tube disposed over the shaft, and the expandable member is enclosed between the shaft and the outer tube when the outer tube is in the first position.

3. The medical device of claim 2, wherein the constricting device applies a constricting force against the expandable member when the constricting device is in the first position, and wherein the constricting force is removed when the constricting device is in the second position.

4. The medical device of claim 1, wherein the constricting device includes an inner tube disposed within the lumen of the shaft, and includes a distal tip that extends distally of a distal end of the shaft.

5. The medical device of claim 4, wherein a proximal portion of the distal tip is coupled to a distal portion of the expandable member.

6. The medical device of claim 4, wherein application of a proximal force on the inner tube causes the expandable member to shorten longitudinally and expand laterally outward.

7. The medical device of claim 1, wherein the shaft includes a slot proximate to the distal portion of the shaft and extending into the lumen of the shaft; and
wherein the constricting device includes a thread extending outwardly from the lumen of the shaft via the slot, and the thread is attached to the distal portion.

8. The medical device of claim 7, wherein a proximal portion of the thread extends through the lumen and a distal portion of the thread is disposed over an outer surface of the distal portion of the shaft and extends about the expandable member.

9. The medical device of claim 8, wherein the distal portion of the thread includes one or more knots linking the thread to the expandable member; and
wherein the one or more knots are configured to be broken or untangled in response to a proximal force applied to the thread.

10. The medical device of claim 1, further including a locking mechanism configured to fix the constricting device relative to the expandable member to thereby maintain the constricting device in at least one of the first position or the second position and the expandable member in at least one of the expanded state or the collapsed state.

11. The medical device of claim 1, further including a pressure source in fluid communication with the lumen, wherein the pressure source is configured to generate suction at the distal portion of the shaft via the side opening such that fluid positioned adjacent to the distal portion are drawn into the lumen through the expandable member.

12. The medical device of claim 1, wherein the expandable member includes a porous body including a plurality of fibers.

13. The medical device of claim 1, wherein the expandable member has a greater diameter in the expanded state than the collapsed state.

14. The medical device of claim 1, wherein the side opening is one of a plurality of side openings extending through the shaft.

15. The medical device of claim 1, wherein the expandable member is fixed to the shaft along an entire length of the expandable member.

16. A medical device, comprising:
a shaft defining a lumen along a longitudinal axis, wherein the shaft includes at least one opening through an outer surface of the shaft, the at least one opening in fluid communication with the lumen;
a porous body disposed over the at least one opening of the shaft and configured to expand laterally outward relative to the longitudinal axis; and
a constricting member engaged against the porous body and movable relative to the porous body, wherein the constricting member is configured to apply a constraining force against the porous body;
wherein the shaft is configured to receive fluid into the lumen via the opening and through the porous body after the constricting member moves the porous body from a collapsed state to an expanded state.

17. The medical device of claim 16, wherein the porous body has a smaller cross-sectional diameter about the longitudinal axis when in the collapsed state than when in the expanded state.

18. The medical device of claim 16, wherein the porous body has a smaller longitudinal length when in the expanded state than when in the collapsed state.

19. The medical device of claim 16, wherein the porous body is a sponge having a porosity that allows passage of fluid through the porous body.

20. A method of withdrawing fluids with a medical device, the medical device including a shaft, an expandable porous body disposed along the shaft, and a constricting device in contact with the expandable porous body; the method comprising:
moving the constricting device relative to the expandable porous body to allow expansion of the expandable porous body about the shaft;
applying suction through the shaft; and
withdrawing fluid through the expandable porous body and into the shaft via one or more openings disposed between the expandable porous body and the shaft.
